(19) United States
(12) Patent Application Publication
(10) Pub. No.: US 2021/0181377 A1
(43) Pub. Date: Jun. 17, 2021

(54) INFORMATION PROCESSING DEVICE, PARAMETER CORRECTION METHOD AND PROGRAM RECORDING MEDIUM

(71) Applicant: NEC CORPORATION, Tokyo (JP)

(72) Inventors: Shinji KASAHARA, Tokyo (JP); Yasuhiro SUGISAKI, Tokyo (JP)

(73) Assignee: NEC CORPORATION, Tokyo (JP)

(21) Appl. No.: 16/078,969

(22) PCT Filed: Feb. 14, 2017
(86) PCT No.: PCT/JP2017/005228

§ 371 Date: Aug. 22, 2018

(30) Foreign Application Priority Data
Feb. 23, 2016 (JP) 2016-031721

(51) Int. Cl. G01W 1/18; G01N 33/24; G01W 1/14

(52) U.S. Cl. CPC G01W 1/18; G01W 1/14; G01N 33/246

(57) ABSTRACT

The present invention evaluates, with high precision, the risk of a landslide disaster. An information processing device 100 is provided with: an estimation unit 110 that estimates a parameter indicating a moisture state of soil of a prescribed site, on the basis of a first piece of data indicating the topography, vegetation or geological features of the site and second piece of data indicating the precipitation amount of the site; a correction formula calculation unit 120 that calculates a correction formula, in regards to a first site that is the site at which a sensor for measuring the parameter is installed, using a parameter measured by the sensor and a first parameter which is the parameter estimated for the first site; and a correction unit 130 that uses the calculated correction formula to correct a second parameter, which is the parameter estimated for a second site, which is a site at which the sensor that measures the parameter is not installed.

### TECHNICAL FIELD

The present invention relates to an information processing device, a parameter correction method, and a program recording medium.

### BACKGROUND ART

PTL 1 discloses a method of calculating moisture content in earth by use of precipitation amount information and conditions regarding, for example, topography, geology, or vegetation of an observation zone, and predicting a risk of a mountain disaster by a calculatio

### ult.

### CITATION LIST

Patent Literature

[PTL 1] Japanese Patent Application Publication No. H10-232286

### SUMMARY OF INVENTION

### Technical Problem

In order to calculate moisture content in earth for each observation zone, the method described in PTL 1 needs an observation means such as a rain gauge for each observation zone. Thus, a large quantity of observation means is needed to realize a high-precision risk prediction by a smaller observation zone. Installation of an observation means needs not only a cost for the observation means itself but also a cost for the installation of the observation means. In addition, there may be a case in which installation of an observation means is difficult because of fear of occurrence of a landslide disaster. In other words, a technique described in PTL 1 has a problem that it is difficult to evaluate a risk of a landslide disaster with high precision.

One exemplary object of the present invention is, as described above, to evaluate a risk of a landslide

### aster with high precision.

### Solution to Problem

An aspect of the invention is an information processing device. The information processing device includes estimation means for estimating a parameter indicating a moisture state of soil at a predetermined site, based on first data indicating topography, vegetation, or geology of the site, and second data indicating a precipitation amount at the site; correction formula calculation means for calculating a correction formula, in regard to a first site where a sensor that measures the parameter is installed, by using a parameter measured by the sensor and a first parameter being estimated for the first site; and correction means for correcting, by using the calculated correction formula, a second parameter estimated for a second site where a sensor that measures the parameter is not installed.

Another aspect of the invention is a parameter correction method. The parameter correction method includes estimating a parameter indicating a moisture state of soil at a predetermined site, based on first data indicating topography, vegetation, or geology of the site, and second data indicating a precipitation amount at the site; calculating a correction formula, in regard to a first site where a sensor that measures the parameter is installed, by using a parameter measured by the sensor and a first parameter estimated for the first site; and correcting, by using the calculated correction formula, a second parameter estimated for a second site where a sensor that measureter is not installed.

Another aspect of the invention is a computer-readable program recording medium. The computer-readable program recording medium records a program causing a computer to execute: processing of estimating a parameter indicating a moisture state of soil at a predetermined site, based on first data indicating topography, vegetation, or geology of the site, and second data indicating a precipitation amount at the site; processing of calculating a correction formula, in regard to a first site where a sensor that measures the parameter is installed, by using a parameter measured by the sensor and a first parameter estimated for the first site; and processing of correcting, by using the calculated correction formula, a second parameter estimated for a second site where a sensor that measures the parameter is not installed.

### Advantageous Effects of Invention

According to the present invention, it is possible to evaluate a risk of a landslide disaster with high precision.

### BRIEF DESCRIPTION OF DRAWINGS

FIG. 1 is a block diagram illustrating one example of a configuration of an information processing device according to a first example embodiment.

FIG. 2 is a flowchart illustrating one example of an operation of the information processing device according to the first example embodiment.

FIG. 3 is a block diagram illustrating one example of a configuration of an evaluation system according to a second example embodiment.

FIG. 4 is a diagram illustrating a first site and a second site in the second example embodiment.

FIG. 5 is a block diagram illustrating a configuration of an evaluation device according to the second example embodiment.

FIG. 6 is a flowchart illustrating one example of a rough operation of the evaluation device according to the second example embodiment.

FIG. 7 is a diagram illustrating one example of a calculation method of a correction formula according to the second example embodiment.

FIG. 8 is a flowchart illustrating one example of correction processing according to a modification example.

FIG. 9 is a block diagram illustrating one example of a configuration of an information processing device according to a modification example.

FIG. 10 is a block diagram illustrating one example of a configuration of an evaluation device according to a modification example.

FIG. 11 is a block diagram illustrating one example of a hardware configuration of a computer device according to a modification example.

### DESCRIPTION OF EMBODIMENTS

### First Example Embodiment

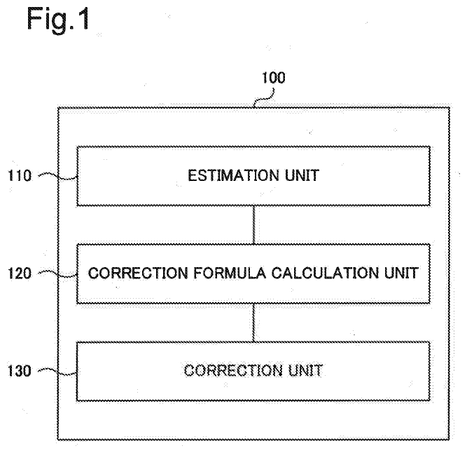

FIG. 1 is a block diagram illustrating a configuration of an information processing device 100 according to a first example embodiment of the present invention. The information processing device 100 includes at least an estimation unit 110, a correction formula calculation unit 120, and a correction unit 130. The information processing device 100 is an information processing device intended to correct a parameter indicating a moisture state of soil.

The estimation unit 110 estimates a parameter indicating a moisture state of soil. The parameter indicating the moisture state of soil is, for example, a saturation degree or moisture content of earth. The saturation degree referred to herein is a ratio of volume of water in a pore to a pore volume of soil. Moreover, the moisture content may be either a volume moisture content (a ratio of volume of moisture to volume of soil) or weight moisture content (a ratio of weight of moisture to weight of soil).

The estimation unit 110 estimates parameters of a plurality of sites. The plurality of sites referred to herein include a site where a sensor (hereinafter referred to as a “soil sensor”.) which measures a parameter is installed, and a site where a soil sensor is not installed. Hereinafter, for convenience of description, a site where a soil sensor is installed is referred to as a “first site”, and a site where a soil sensor is not installed is referred to as a “second site”.

The first site and the second site are each, for example, a region (also referred to as a “grid” or a “area grid” in general.) obtained by dividing an area to be evaluated into predetermined sizes. Specifically, the first site and the second site are quadrates of one kilometer square, five kilometers square, or the like, but are not limited to a particular shape or a particular size. Moreover, the numbers of first sites and second sites are not limited to a particular number.

The estimation unit 110 estimates parameters of these sites, based on a plurality of pieces of data. The estimation unit 110 estimates a parameter (first parameter) of the first site and a parameter (second parameter) of the second site, based on data (hereinafter referred to as “first data”.) indicating topography, vegetation, or geology of the site, and data (hereinafter referred to as “second data”.) indicating a precipitation amount of the site.

The first data represent, for example, a height difference between adjacent sites. Alternatively, the first data may represent presence or absence, or a kind of a plant (coniferous forest, broadleaf forest, field of grass, or the like) at the site. Otherwise, the first data may represent composition of soil of the site.

The second data are typically a predictive value of a precipitation amount. For example, in Japan, the Meteorological Agency announces a predictive value of a precipitation amount in each grid unit of one kilometer square (short-term precipitation forecast). The estimation unit 110 may use, as the second data, such a predictive value provided from an external institution or enterprise, or use, as the second data, a predictive value used in a simulation or the like. Note that, the second data may be a precipitation amount observed by a weather radar or the like.

Note that, an estimation algorithm of a parameter used by the estimation unit 110 is not limited to a particular algorithm. However, the estimation unit 110 may estimate a parameter by use of the same estimation algorithm for the first site and the second site. It can be said that using the same estimation algorithm for the first site and the second site increases likelihood of a certain tendency produced in an error occurring between parameters estimated in these sites.

The correction formula calculation unit 120 calculates a correction formula of a parameter. In regard to the first site, the correction formula calculation unit 120 calculates a correction formula, by use of the parameter (i.e., estimated value) estimated by the estimation unit 110, and the parameter (i.e., actually measured value) measured by the soil sensor. The correction formula calculation unit 120 calculates a correction formula which corrects the estimated value in such a way that a difference between the estimated value and the actually measured value decreases.

The correction unit 130 corrects a parameter. The correction unit 130 corrects the parameter of the second site among the parameters estimated by the estimation unit 110, using the correction formula calculated by the correction formula calculation unit 120. In other words, the correction unit 130 corrects the parameter estimated for the second site (where a soil sensor is not installed), using the correction formula calculated based on the parameter of the first site (where the soil sensor is installed).

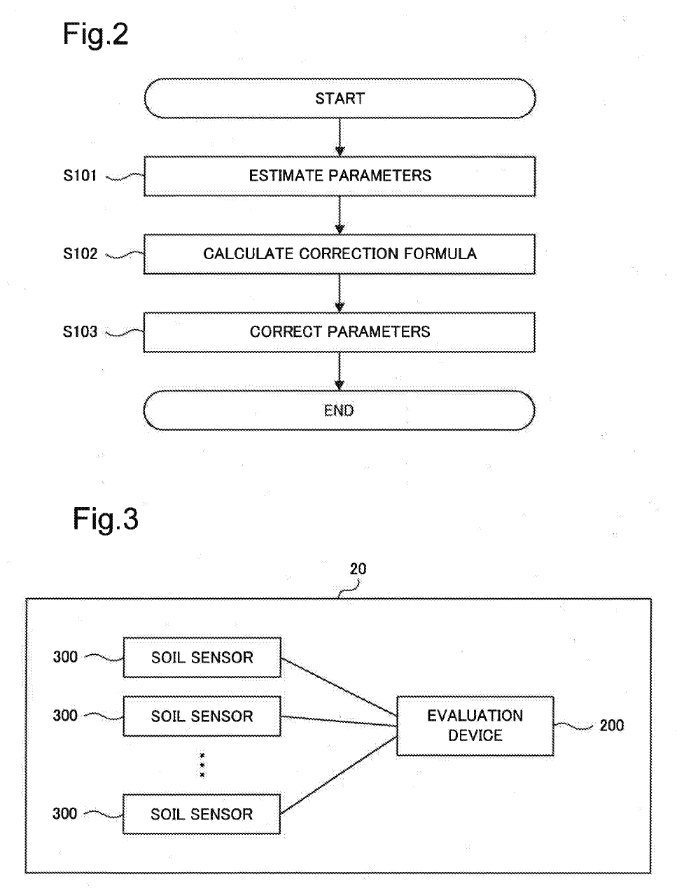

FIG. 2 is a flowchart illustrating one example of an operation of the information processing device 100 according to the present example embodiment. Note that, the information processing device 100 may vary an execution order of steps illustrated in FIG. 2 without affecting an action and advantageous effects.

Prior to the estimation of the parameters, the estimation unit 110 acquires the first data and the second data for the first site and the second site, respectively. In this instance, the estimation unit 110 may acquire data from a storage medium included in the local device, or may acquire data from another device. Having acquired the first data and the second data, the estimation unit 110 estimates parameters, based on these pieces of data (step S101).

The estimation unit 110 supplies the parameter of the first site to the correction formula calculation unit 120 among the estimated parameters, and supplies the parameter of the second site to the correction unit 130. Alternatively, the estimation unit 110 may write these parameters into a predetermined storage medium in such a way that the correction formula calculation unit 120 and the correction unit 130 are able to read the parameters.

Prior to the calculation of the correction formula, the correction formula calculation unit 120 acquires a parameter measured by the soil sensor. Hereinafter, in order to distinguish between the parameter estimated in step S101 and the parameter measured by the soil sensor, the former is also referred to as an “estimated value”, and the latter is also referred to as an “actually measured value”.

Using the estimated value of the first site and the actually measured value of the site, the correction formula calculation unit 120 calculates a correction formula intended to correct the estimated value of the second site (step S102). The correction formula calculation unit 120 supplies the calculated correction formula to the correction unit 130, or writes the calculated correction formula into a predetermined storage medium.

Using the estimated value of the second site among the estimated values estimated in step S101, and the correction formula calculated in step S102, the correction unit 130 corrects the estimated value (step S103). The corrected estimated value (i.e., parameter) is used, for example, for calculation of a safety factor in the information processing device 100 or some other device.

As a consequence, according to the information processing device 100 in the present example embodiment, it is possible to correct a parameter for the second site where a soil sensor is not installed, using the correction formula calculated based on a relation between the estimated value and the actually measured value of the parameter at the first site. Therefore, according to the information processing device 100, as compared to the case where such a correction is not executed, it is possible to improve precision of a parameter, and execute, with high precision, an evaluation of a risk of a landslide disaster using the parameter, even when the installation number of soil sensors is limited.

Furthermore, the information processing device 100 is able to improve precision of the parameter of the second site, and can therefore exert an appendant action and advantageous effects of being able to lessen the installation number of sensors, and reducing a size of each site (grid).

### Second Example Embodiment

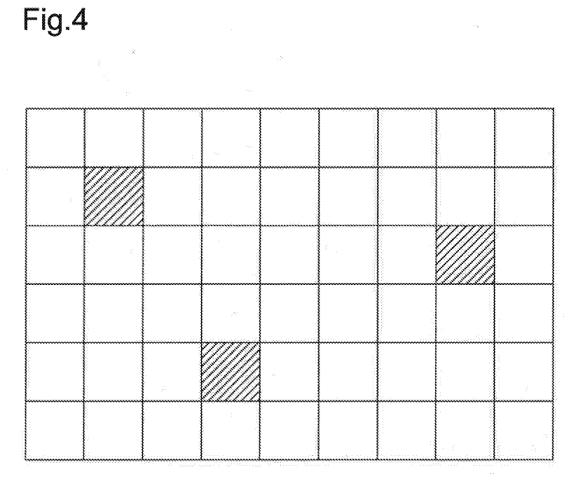

FIG. 3 is a block diagram illustrating a configuration of an evaluation system 20 according to a second example embodiment of the present invention. The evaluation system 20 includes an evaluation device 200, and a soil sensor 300. Note that, among terms used in the present example embodiment, a term which is also used in the first example embodiment is used in a sense similar to that in the first example embodiment.

The evaluation system 20 is a system which evaluates a safety factor of a predetermined area. The predetermined area referred to herein is, for example, an area where a landslide disaster such as a slope failure tends to occur. Moreover, the safety factor referred to herein is a safety factor used in a slope stability analysis (i.e., safety factor of a slope).

The soil sensor 300 is installed in a predetermined site (first site) among areas to be evaluated. The soil sensor 300 measures and outputs a parameter indicating a moisture state of soil. The number of soil sensors 300 needs only to be any number of 1 or more, and is not limited to a particular number. However, the number of soil sensors 300 is more than one in the following description.

FIG. 4 is a diagram illustrating a first site and a second site in the present example embodiment. In the present example embodiment, an area to be evaluated is divided into grids of a predetermined size. The first site is equivalent to a grid where the soil sensor 300 is installed among these grids. The second site is equivalent to a grid where the soil sensor 300 is not installed among these grids. In FIG. 4, the first site is indicated with hatching. In other words, the first site and the second site are indicated in different forms according to FIG. 4.

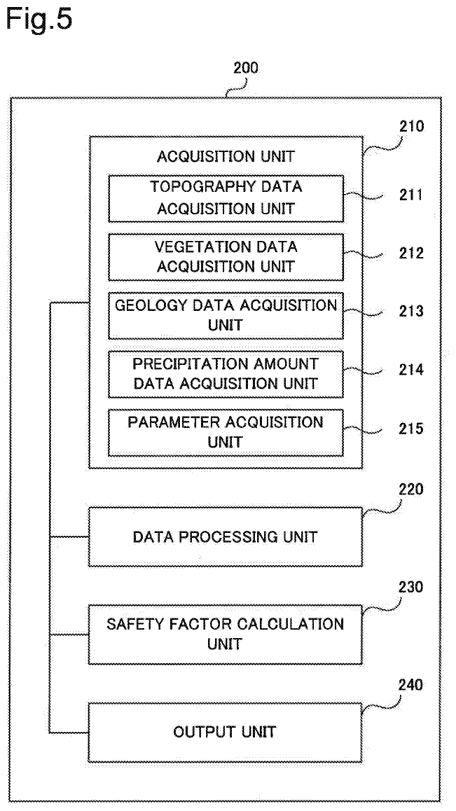

FIG. 5 is a block diagram illustrating a configuration of the evaluation device 200. The evaluation device 200 includes an acquisition unit 210, a data processing unit 220, a safety factor calculation unit 230, and an output unit 240.

The acquisition unit 210 acquires a plurality of kinds of data. More specifically, the acquisition unit 210 includes a topography data acquisition unit 211, a vegetation data acquisition unit 212, a geology data acquisition unit 213, a precipitation amount data acquisition unit 214, and a parameter acquisition unit 215.

The topography data acquisition unit 211 acquires topography data indicating topography of each grid. The topography data indicates, for example, an altitude of each grid. Alternatively, the topography data may indicate a height difference between adjacent grids, or indicate a direction of flow of water (based on the height difference) by a bearing.

The vegetation data acquisition unit 212 acquires vegetation data indicating vegetation of each grid. The vegetation data indicates, for example, whether or not each grid has vegetation. Generally, soil which does not have vegetation tends to increase and decrease in moisture content in earth, as compared with soil which has vegetation. Moreover, the tendency of changing of moisture content in earth also varies depending on the kind of vegetation. Thus, the vegetation data may indicate the kind of vegetation of each grid, or may be a numerical form of a tendency to increase and decrease in moisture content based on a difference of vegetation.

The geology data acquisition unit 213 acquires geology data indicating geology of each grid. The geology data indicates, for example, composition of soil of each grid. Alternatively, the geology data may be a numerical form of a tendency to increase and decrease in moisture content in each soil based on a difference of composition of soil of each grid.

The precipitation amount data acquisition unit 214 acquires precipitation amount data indicating a precipitation amount of each grid.

The precipitation amount data indicates, for example, a predictive value of a precipitation amount of each grid after a predetermined time. The precipitation amount data acquisition unit 214 may acquire precipitation amount data at a plurality of time points (e.g., a rainfall predictive value per hour from the current time to four hours later).

The topography data, the vegetation data, and the geology data are equivalent to one example of the first data described above. On the other hand, the precipitation amount data are equivalent to one example of the second data described above. The acquisition unit 210 may acquire all or only one of the topography data, the vegetation data, and the geology data.

The parameter acquisition unit 215 acquires the parameter output from the soil sensor 300. Note that, the parameter acquisition unit 215 does not need to directly acquire the parameter from the soil sensor 300. For example, the parameter acquisition unit 215 may read a parameter output from the soil sensor 300 and stored in a predetermined storage device.

The topography data acquisition unit 211, the vegetation data acquisition unit 212, the geology data acquisition unit 213, the precipitation amount data acquisition unit 214, and the parameter acquisition unit 215 may have the same acquisition path of data, or different acquisition paths of data. In other words, the acquisition unit 210 may include a configuration which acquires data via a network, and a configuration which reads data stored in a storage device. The acquisition unit 210 may acquire data via a network differing from data to data.

The data processing unit 220 corresponds to the information processing device 100 according to the first example embodiment. In other words, the data processing unit 220 includes a configuration equivalent to the estimation unit 110, the correction formula calculation unit 120, and the correction unit 130. Using the data acquired by the acquisition unit 210, the data processing unit 220 executes estimation of a parameter, calculation of a correction formula, and correction of the parameter. The data processing unit 220 outputs a corrected parameter of the second site, and a parameter (actually measured value) of the first site.

Using the parameter output from the data processing unit 220, the safety factor calculation unit 230 calculates a safety factor of each grid. The safety factor calculation unit 230 calculates a safety factor corresponding to each grid by substituting a parameter for a predetermined definitional equation (stability analysis equation) which calculates a safety factor. Note that, the stability analysis equation intended to calculate a safety factor needs only to be an equation which makes it possible to uniquely obtain a safety factor by the parameter output from the data processing unit 220, and is not limited to a particular equation.

As the stability analysis equation in the slope stability analysis, there are known stability analysis equations by Fellenius method, modified Fellenius method, Bishop method, and Janbu method. Moreover, various stability analysis equations which are applications or modifications of the above stability analysis equations are known. The safety factor calculation unit 230 calculates a safety factor from a parameter, based on such a stability analysis equation.

Note that, the safety factor calculation unit 230 may calculate only a safety factor of the second site, and does not need to calculate a safety factor of the first site. In this case, the safety factor of the first site may be calculated in some other way by a means other than the safety factor calculation unit 230. In other words, the parameter of the first site may be used only to calculate a correction formula in the evaluation device 200.

The output unit 240 outputs information corresponding to the safety factor calculated by the safety factor calculation unit 230. The output unit 240 has, for example, a display device such as a liquid crystal display.

In this case, the output unit 240 may separately display a grid of an area to be evaluated with a color corresponding to the safety factor thereof, or may display a safety factor of each grid in a list form. Alternatively, the output unit 240 may highlight a grid having a safety factor less than a predetermined threshold (e.g., “1.06”), or may display a predetermined message (warning sentence or the like) when a grid having a safety factor less than the predetermined threshold exists.

The output unit 240 may output information corresponding to the calculated safety factor in a way different from display. For example, the output unit 240 may have a speaker, and reproduce a warning sound or the like, or send information corresponding to a safety factor to some other device.

The configuration of the evaluation system 20 is as above. Under such a configuration, the evaluation device 200 calculates a safety factor, based on a parameter. Prior to the calculation of the safety factor, the evaluation device 200 acquires necessary data such as a parameter (actually measured value). Specifically, the evaluation device 200 operates as below.

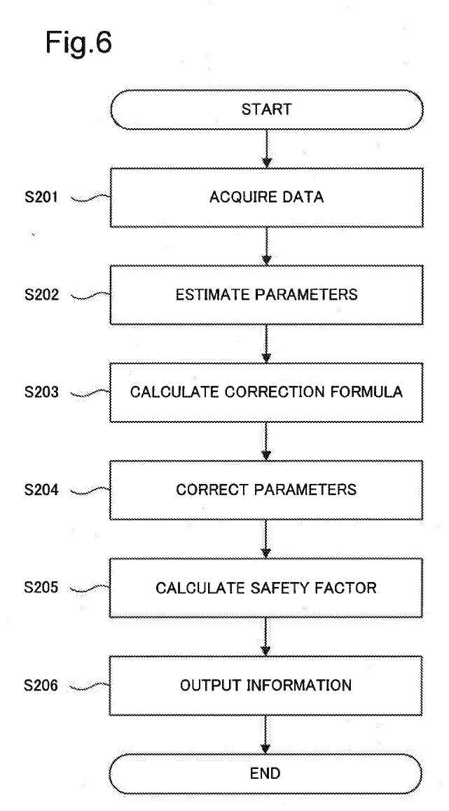

FIG. 6 is a flowchart illustrating one example of a rough operation of the evaluation device 200. In the operation illustrated in FIG. 6, the acquisition unit 210 first acquires necessary data (step S201). Specifically, the acquisition unit 210 acquires first data, second data, and a parameter. Although processing of acquiring these data is illustrated as a single step for convenience of description in FIG. 6, the processing may be executed at different timing for each data.

When the necessary data are prepared, the data processing unit 220 estimates a parameter of each grid, based on the first data and the second data (step S202). Then, the data processing unit 220 calculates a correction formula, based on an estimated value and an actually measured value of a parameter of a grid equivalent to the first site (step S203). The data processing unit 220 corrects an estimated value of a parameter of a grid equivalent to the second site by use of the correction formula calculated in step S203 (step S204). Hereinafter, the parameter corrected in step S204 is also referred to as a “corrected value” in order to distinguish from other parameters.

The safety factor calculation unit 230 calculates a safety factor of each grid, based on the parameter (step S205). Specifically, the safety factor calculation unit 230 calculates a safety factor of the grid equivalent to the first site, based on the actually measured value of the parameter, and calculates a safety factor of the grid equivalent to the second site, based on the corrected value of the parameter. The output unit 240 outputs (displays or in some other way) information corresponding to the safety factor thus calculated (step S206).

The estimation of the parameter in step S202 is specifically conducted as below. The data processing unit 220 estimates a parameter by estimating a water balance (inflow and outflow of moisture) in each grid. The data processing unit 220 estimates a parameter by simulating in such a way that moving moisture is separated into groundwater (water underground) and surface water (water on the surface). Note that, the groundwater referred to herein refers to moisture contained in soil in an unsaturated zone (i.e. soil water).

For example, the data processing unit 220 simulates flow of surface water of each grid as below. The flow of surface water is represented by equation (1.1) below in accordance with a continuity equation. Moreover, equation (1.2) below is satisfied by a momentum equation of a diffusion wave.

$$\begin{matrix}[ Expression 1 ] & \\ \frac{\partial {h}_{s}}{\partial t} = - ∇ \cdot ( {h}_{s} v ) - {q}_{s} & ( 1.1 ) \\ [ Expression 2 ] & \\ {i}_{g} + \frac{\partial {h}_{s}}{\partial x} {cos}^{2} β + \frac{{n}^{2} {v}^{2}}{{R}^{4 / 3}} = 0 & ( 1.2 )\end{matrix}$$

Herein,
R: hydraulic radius,
hₛ: water depth,
i_{g}: riverbed gradient,
n: Manning's roughness coefficient,
qₛ: inflow amount (of surface water),
v: flow velocity,
β: angle of slop, and
x: moving direction of water (horizontal direction).

Note that, the flow velocity v in equation (1.1) is a vector quantity having components of a moving direction (downflow direction) of surface water on the surface and a water depth direction (permeating direction). However, the component of the flow velocity v in the water depth direction is small to a negligible degree as compared with the component on the surface in the moving direction. Thus, the flow velocity v in and after equation (1.2) is represented as a scalar quantity indicating a size of the component on the surface in the moving direction between the two components described above.

Herein, when an approximation by equation (1.3) below is applied to equation (1.2), the flow velocity v is represented by equation (1.4) below.

$$\begin{matrix}[ Expression 3 ] & \\ {\vert {i}_{g} + \frac{\partial {h}_{s}}{\partial x} {cos}^{2} β \vert}^{1 / 2} \approx {\vert {i}_{g} \vert}^{1 / 2} & ( 1.3 ) \\ [ Expression 4 ] & \\ v = - \frac{{R}^{2 / 3}}{n {\vert {i}_{g} \vert}^{1 / 2}} ( {i}_{g} + \frac{\partial {h}_{s}}{\partial x} {cos}^{2} β ) & ( 1.4 )\end{matrix}$$

Furthermore, the data processing unit 220 simulates flow of groundwater of each grid as below. The flow of groundwater is represented by equation (2.1) below in accordance with a continuity equation. Moreover, equation (2.2) below is satisfied by Darcy's law.

$$\begin{matrix}[ Expression 5 ] & \\ \frac{\partial ( {p}_{w} φ {S}_{w} )}{\partial t} = - ∇ \cdot {F}_{w} - {ρ}_{w} {q}_{w} & ( 2.1 ) \\ [ Expression 6 ] & \\ {F}_{w} = {ρ}_{w} {u}_{w} = - \frac{{ρ}_{w} {k}_{rw} k}{{μ}_{w}} ∇ ( {P}_{w} + {ρ}_{w} g z ) & ( 2.2 )\end{matrix}$$

Herein,
F_{w}: mass flow rate,
P_{w}: water pressure,
S_{w}: degree of saturation,
g: acceleration of gravity,
k: permeability coefficient,
k_{rw}c permeability coefficient,
q_{w}inflow amount (of groundwater),
u_{w}Darcy velocity,
ρ_{w}: density of water,
ϕ: rate of porosity of soil,
μ_{w}: viscosity of water,
z: water level from bedrock surface, and
t: time.

The mass flow rate F_{w} is considered herein as the mass of water flowing out from a grid in a particular direction (x-direction) per unit time. The water level z changes according to a moisture state in earth, and can therefore also be expressed by a function of a moisture state (e.g., moisture content). Moreover, the mass flow rate F_{w} and the Darcy velocity u_{w} are vector quantities herein.

Using these equations, the data processing unit 220 estimates a parameter (herein, a degree of saturation). Details of estimation processing vary depending on the kind of first data (topography data, vegetation data, or geology data) as indicated below.

(When topography data are used)

When topography data are used, the data processing unit 220 determines the inflow amount qₛ of equation (1.1), based on precipitation amount data. Specifically, the data processing unit 220 determines the inflow amount qₛ of a grid (hereinafter, referred to as a “grid in the most upstream part”.) located at a position higher than any of a plurality of adjacent grids, based on precipitation amount data of the grid. In other words, the data processing unit 220 considers that the grid in the most upstream part does not have any inflow from other grids, and the inflow amount qₛ depends on only the precipitation amount of the grid. Moreover, herein, the hydraulic radius R, the riverbed gradient i_{g}, the roughness coefficient n, and the angle of slop β are uniquely given by topography data.

Accordingly, unknowns in equation (1.1) and equation (1.4) are only the water depth hₛ and the flow velocity v. The data processing unit 220 solves equation (1.1) and equation (1.4) for a grid in the most upstream part, based on the precipitation amount data, and calculates a water depth hₛ and a flow velocity v.

Furthermore, for a grid other than a grid in the most upstream part, the data processing unit 220 considers, as the inflow amount qₛ, a sum of a precipitation amount for the grid, and an outflow amount as surface water from a grid adjacent to the grid and located at a position higher than the grid. The outflow amount as surface water to other grids is specified based on the water depth hₛ and the flow velocity v. Moreover, surface water having the water depth hₛ that does not permeate earth and remains on the surface is estimated to flow out to a downstream grid at the flow velocity v, based on the riverbed gradient i_{g}. Note that, whether or not each grid is equivalent to the most upstream part may be determined in advance, or may be specified based on topography data. By the specification of the inflow amount qₛ, the data processing unit 220 is able to calculate the water depths hₛ and the flow velocities v of other grids in a manner similar to the case of the most upstream part.

Moreover, the data processing unit 220 determines the mass flow rate F_{w} and the inflow amount q_{w}, based on precipitation amount data.

Specifically, the data processing unit 220 determines the mass flow rate F_{w} and the inflow amount q_{w} of a grid in the most upstream part, based on the precipitation amount data of the grid, and an outflow amount as surface water from the grid. The data processing unit 220 considers that moisture other than rain does not flow into a grid in the most upstream part, and uses, as the inflow amount q_{w}, a value in which the outflow amount as surface water is subtracted from the precipitation amount indicated by the precipitation amount data. In addition, at the start of calculation, the data processing unit 220 calculates the mass flow rate F_{w} of a grid in the most upstream part by use of equation (2.2) with z=0 (or a predetermined value other than 0).

The data processing unit 220 calculates the inflow amount q_{w} into a grid other than a grid in the most upstream part, based on a precipitation amount for the grid, and an outflow amount (i.e. the mass flow rate F_{w}) as soil water from a grid adjacent to the grid. For example, the data processing unit 220 calculates the inflow amount q_{w} into a grid adjacent to a grid in the most upstream part, based on a precipitation amount for the grid, and the mass flow rate F_{w} of a grid in the most upstream part. Thus, the data processing unit 220 is able to calculate the inflow amount q_{w} of a grid located at a lower position, based on a precipitation amount for the grid, and the mass flow rate F_{w} of a grid located at a position higher than the grid. In addition, at the start of calculation, the data processing unit 220 calculates the mass flow rate F_{w} of a grid other than a grid in the most upstream part by use of equation (2.2) with z=0 (or a predetermined value other than 0).

Using the mass flow rate F_{w} and the inflow amount q_{w} calculated as above, the data processing unit 220 calculates water pressure P_{w} and a degree of saturation S. In this case, the acceleration of gravity g, the permeability coefficient k, the specific permeability coefficient k_{rw}, the Darcy velocity u_{w} (determined by the permeability coefficient k), the density ρ_{w}, the rate of porosity ϕ, and the viscosity μ_{w} are fixed values determined in advance. In other words, unknowns in equation (2.1) and equation (2.2) are only the water pressure P_{w} and the degree of saturation S_{w}.

(When vegetation data are used)

When vegetation data are used, the data processing unit 220 determines the inflow amount qₛ of equation (1.1), based on precipitation amount data, as in the case where topography data are used. Herein, the hydraulic radius R, the riverbed gradient i_{g}, the roughness coefficient n, and the angle of slop β may be fixed values determined in advance, but may be uniquely given by topography data.

Accordingly, unknowns in equation (1.1) and equation (1.4) are only the water depth hₛ and the flow velocity v. Thus, when vegetation data are used as well, the data processing unit 220 is able to calculate a water depth hₛ and a flow velocity v, as in the case where topography data are used.

Moreover, the data processing unit 220 determines the mass flow rate F_{w} and the inflow amount q_{w}, based on precipitation amount data and the vegetation data. In this case, the permeability coefficient k, the specific permeability coefficient k_{rw}, the Darcy velocity u_{w}, and the Manning's roughness coefficient n are uniquely given based on the vegetation data. Generally, the permeability coefficient k and the specific permeability coefficient k_{rw} vary depending on the kind of vegetation, and the permeability coefficient k tends to be higher when a distribution ratio of a root system is higher. Moreover, the Manning's roughness coefficient n tends to be higher when density (size and a degree of growth) of vegetation is higher. In addition, it is assumed that the acceleration of gravity g, the density ρ_{w}, the viscosity μ_{w}, and the rate of porosity ϕ are fixed values determined in advance. Accordingly, the data processing unit 220 is able to calculate the mass flow rate F_{w} and the inflow amount q_{w} from equation (2.1) and equation (2.2), and is able to calculate the water pressure P_{w} and the degree of saturation S_{w} by use of the calculated mass flow rate F_{w} and inflow amount q_{w}.

(When geology data are used)

When geology data are used, the data processing unit 220 determines the inflow amount qₛ of equation (1.1), based on precipitation amount data, as in the case where vegetation data are used. Herein, the hydraulic radius R, the riverbed gradient i_{g}, the roughness coefficient n, and the angle of slop β may be fixed values determined in advance, but may be uniquely given by geology data. When geology data are used as well, the data processing unit 220 is able to calculate a water depth hₛ and a flow velocity v, as in the case where vegetation data are used.

Moreover, the data processing unit 220 determines the mass flow rate F_{w} and the inflow amount q_{w}, based on precipitation amount data and geology data. Specifically, in this case, the permeability coefficient k, the specific permeability coefficient k_{rw}, the Darcy velocity u_{w}, the density ρ_{w}, and the rate of porosity ϕ are uniquely given based on the geology data. In addition, it is assumed that the acceleration of gravity g, the Darcy velocity u_{w}, the density ρ_{w}, and the rate of porosity ϕ are fixed values determined in advance. Accordingly, the data processing unit 220 is able to calculate the mass flow rate F_{w} and the inflow amount q_{w} from equation (2.1) and equation (2.2), and is able to calculate the water pressure P_{w} and the degree of saturation S_{w} by use of the calculated mass flow rate F_{w} and inflow amount q_{w}.

Estimation processing of a parameter is as above. Then, calculation of a correction formula in step S203 is specifically conducted as below. The data processing unit 220 derives a regression expression of calculating a sensor value with the degree of saturation S_{w} as a variable by acquiring, under conditions with a plurality of different degree of saturations, a degree of saturation S_{w} calculated at the first site, and a sensor value measured by a sensor of the first site. For example, the data processing unit 220 is able to calculate the degree of saturation S_{w} by using a soil moisture meter which measures a moisture rate, and can therefore derive a correction formula of the degree of saturation by using actually measured value of the degree of saturation calculated from the sensor value. Moreover, by previously deriving a relational expression of the sensor value and the water pressure from an experiment or the like, the data processing unit 220 is able to obtain a relation of the water pressure P_{w} and water pressure estimated from the sensor value, and is able to also derive a correction formula thereof. The data processing unit 220 is also able to derive a correction formula of the water pressure by use of the water pressure P_{w} and a water pressure gauge (or a water gauge).

When there are a plurality of first sites, i.e., grids where the soil sensors 300 are installed, the data processing unit 220 calculates a correction formula for each of the first sites. Using at least one of the plurality of calculated correction formulas, the data processing unit 220 corrects a parameter of a second site, i.e., a grid where the soil sensor 300 is not installed.

Using the correction formula of a site close in distance to the second site among the plurality of correction formulas calculated for the plurality of first sites, the data processing unit 220 may correct a parameter of the second site. For example, when correcting a parameter of a second site, the data processing unit 220 uses a correction formula of a site having the shortest distance to the second site among a plurality of first sites.

Alternatively, using a correction formula of a site similar in at least one of topography, vegetation, and geology to a second site among a plurality of correction formulas calculated for a plurality of first sites, the data processing unit 220 may correct a parameter of the second site. For example, the data processing unit 220 calculates, in accordance with a predetermined algorithm, similarity to a second site in regard to topography, vegetation, and geology for a plurality of first sites, and corrects the parameter of the second site by use of a correction formula of the first site which has the highest similarity calculated (i.e., which is most similar).

Alternatively, the data processing unit 220 may calculate a weighted correction formula by a weighted arithmetical operation using a plurality of correction formulas calculated for a plurality of first sites. The data processing unit 220 may vary a weight in the weighted arithmetical operation depending on distances between a plurality of first sites and a second site, or depending on a difference in at least one of topography, vegetation, and geology between a plurality of first sites and a second site. Using the weighted correction formula calculated by the weighted arithmetical operation, the data processing unit 220 corrects a parameter of the second site.

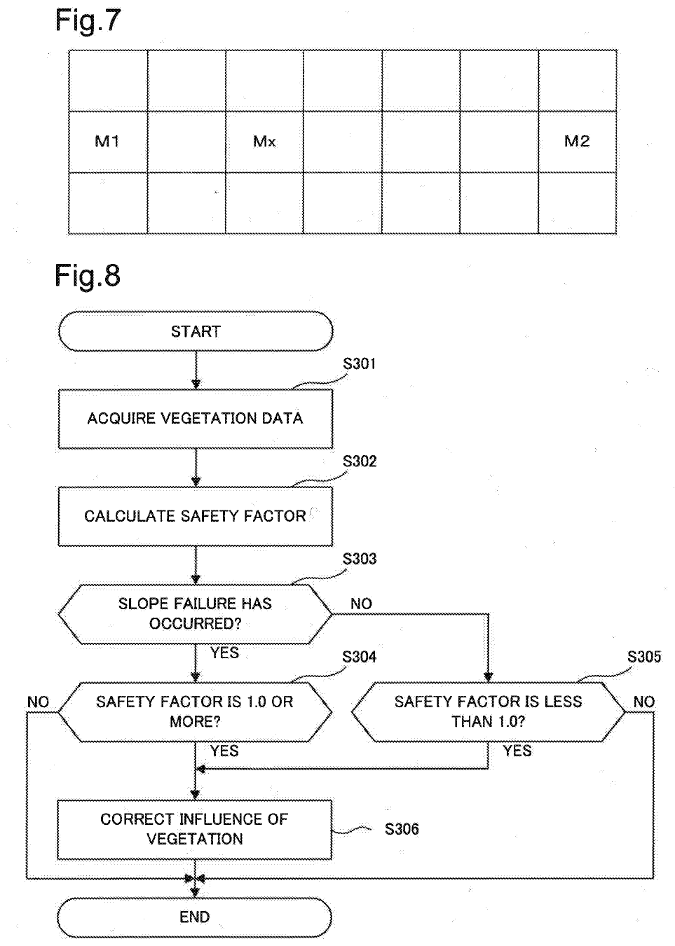

FIG. 7 is a diagram illustrating one example of a calculation method of a correction formula. In this example, it is assumed that for convenience of description, a gradient, vegetation, and geology are uniform, and the size of each grid is the same. Grids M1 and M2 are equivalent to first sites. Moreover, it is assumed that a correction formula of the grid M1 is f_{M1}(m), and a correction formula of the grid M2 is f_{M2}(m). Herein, m is a parameter indicating a moisture state of soil.

A grid Mx is equivalent to a second site. The grid Mx has a distance of two grids to the grid Ml, and a distance of four grids to the grid M2. Thus, the data processing unit 220 calculates a correction formula f_{MX}(m) of the grid Mx as below.

$${f}_{M x} ( m ) = \frac{[ ( 2 + 4 ) - 2 ] {f}_{M 1} ( m ) + [ ( 2 + 4 ) - 4 ] {f}_{M 2} ( m )}{( 2 + 4 )} = \frac{2 {f}_{M 1} ( m ) + {f}_{M 2} ( m )}{3} \qquad [ Expression 7 ]$$

Calculation processing of a correction formula is as above. Then, calculation of a safety factor in step S205 is specifically conducted as below. By use of a predetermined stability analysis equation, the data processing unit 220 calculates a safety factor by use of the estimated and corrected parameter.

For example, a safety factor Fs by Fellenius method can be represented by equation (3.1) below. Herein, c, W, u, and ϕ are variables representing viscosity, weight, pore pressure, and an internal frictional angle of a clay lump, respectively. Moreover, a represents a tilt angle of a slope. Further, 1 represents length of a sliding surface of a divisional piece (slice) obtained by dividing a slope in a vertical direction. For convenience of description, it is assumed that the tilt angle a and the sliding surface length 1 are constants herein.

$$\begin{matrix}[ Expression 8 ] & \\ Fs = \frac{\sum \{ c l + ( W cos α - ul ) tan ϕ \}}{\sum W sin α} & ( 3.1 )\end{matrix}$$

Furthermore, the safety factor Fs by modified Fellenius method can be represented by, for example, equation (3.2) below. Herein, b represents width of a slice. It is assumed that the slice width b is a constant herein.

$$\begin{matrix}[ Expression 9 ] & \\ Fs = \frac{\sum \{ c l + ( W - u b ) cos α \cdot tan ϕ \}}{\sum W sin α} & ( 3.2 )\end{matrix}$$

Herein, the viscosity c, the weight W, the pore pressure u, and the internal frictional angle ϕ each change according to moisture content in earth. Therefore, each of these variables can be represented as a function of moisture content. For example, equation (3.1) is represented by equation (3.3) below when the viscosity c, the weight W, the pore pressure u, and the internal frictional angle ϕ are replaced with functions c(m), W(m), u(m), and ϕ(m) of moisture content m, respectively. In other words, the safety factor Fs can be uniquely specified by being given the moisture content m. Such replacement is also possible in equation (3.2) in a similar way.

$$\begin{matrix}[ Expression 10 ] & \\ Fs = \frac{\sum \{ c ( m ) l + ( W ( m ) cos α - u ( m ) l ) tan ϕ ( m ) \}}{\sum W ( m ) sin α} & ( 3.3 )\end{matrix}$$

Note that, the functions c(m), W(m), u(m), and ϕ(m) may vary from soil to soil. The functions c(m), W(m), u(m), and ϕ(m) may be found in advance, based on the variables thereof and an actually measured value of moisture content, or may be estimated by a simulation or the like.

Both the moisture content m and the degree of saturation S_{w} vary depending on a moisture state in earth. The degree of saturation S_{w} increases depending on an increase of the moisture content m. Thus, the degree of saturation S_{w} can be described as a monotone increasing function of the moisture content m. Therefore, the safety factor Fs can be uniquely specified from not only the moisture content m but also the degree of saturation S_{w}.

Note that, the pore pressure u may be replaced with the function u(m), but may be replaced with the water pressure P_{w} specified by equation (2.2).

As a consequence, according to the evaluation system 20 in the present example embodiment, it is possible to calculate a safety factor for a second site where the soil sensor 300 is not installed, based on a parameter corrected by use of a correction formula calculated based on a parameter at a first site. Therefore, according to the evaluation system 20, as compared to the case where such a correction is not performed, precision of a safety factor can be improved even when the installation number of soil sensors 300 is limited, and an evaluation of a risk of a landslide disaster using this safety factor can be executed with high precision.

### MODIFICATION EXAMPLES

Example embodiments of the present invention are not limited to the first example embodiment and the second example embodiment described above. Example embodiments of the present invention may include a form in which a modification or an application comprehensible to a person skilled in the art is applied to the disclosure by the present description. For example, example embodiments of the present invention may include modification examples described below. Moreover, an example embodiment of the present invention may be an example embodiment in which an example embodiment and a modification example described in the present description are suitably combined as needed. For example, a matter described by use of a particular example embodiment is also applicable to other example embodiments.

### Modification Example 1

A parameter indicating a moisture state of soil is not limited to the example described above. For example, moisture content has a correlation with a damping factor of a vibration waveform in soil. Therefore, when a correlation between moisture content and a damping factor can be found, a stability analysis equation can be described as a function of the damping factor.

### Modification Example 2

A safety factor used for an evaluation of a risk of a landslide disaster is not limited to the example described above. The data processing unit 220 may vary a stability analysis equation intended to calculate a safety factor, depending on the kind of first data.

For example, in a slope stability analysis model by Simons et al., vegetation has an influence on a change of a safety factor. A stability analysis equation of this model can be represented by equation (4.1) below. When vegetation data are used as first data, the data processing unit 220 may calculate a safety factor in accordance with equation (4.1).

$$\begin{matrix}[ Expression 11 ] & \\ Fs = \frac{{c}_{s} ( m ) + {c}_{r} + A {cos}^{2} β tan ϕ ( m )}{B sin β cos β} & ( 4.1 ) \\ where & \\ A = {q}_{0} + ( {γ}_{sat} - {γ}_{w} ) ( h ( m ) - z ) + {γ}_{s} ( H - h ( m ) ) & \\ B = {q}_{0} + {γ}_{sat} ( h ( m ) - z ) + {γ}_{s} ( H - h ( m ) ) & \end{matrix}$$

Herein,
Fs: safety factor,
cₛ (m): adhesion of earth,
cᵣ: adhesion by root system,
ϕ(m): internal frictional angle of earth,
γₛₐₜ: wet specific weight of earth,
γ_{w}: specific weight of water,
γₛ: specific weight of earth,
H: surface earth layer from bedrock surface,
h(m): water level from bedrock surface,
z: height from bedrock surface to sliding surface,
β: slope angle, and
q₀: upper load by vegetation.

Note that, cₛ(m), ϕ(m), and h(m) are functions of the moisture content m, as in the case of equation (3.3).

Among these, values which change under the influence of vegetation are the adhesion cᵣ and the upper load q₀. For example, in the case where vegetation data represent whether or not vegetation is present, the adhesion cᵣ and the upper load q₀ may be positive constants when vegetation is present, and may be 0 when vegetation is not present.

When calculating the safety factor in this way, the data processing unit 220 may correct the influence of vegetation. For example, the data processing unit 220 is able to correct the influence of vegetation (e.g., the values of the adhesion cᵣ and the upper load q₀) by comparing a safety factor obtained by a simulation in a certain slope with a slope status (actual situation) in the slope.

FIG. 8 is a flowchart illustrating correction processing according to the present modification example. In this example, the data processing unit 220 acquires vegetation data (step S301), and calculates a safety factor, based on the vegetation data (step S302). In this instance, the data processing unit 220 executes estimation and correction of a parameter as in the second example embodiment.

The data processing unit 220 simulates a safety factor of a slope to be evaluated at a certain time, and compares the safety factor with an actual situation of the slope to be evaluated at the certain time. Specifically, the data processing unit 220 branches processing depending on whether or not a slope failure has occurred in the slope to be evaluated (step S303). Whether or not a slope failure has occurred does not need to be determined by the data processing unit 220 itself, and needs only to be visually checked by a person, and a check result needs only to be input to the evaluation device 200.

When a slope failure has occurred (step S303: YES), the data processing unit 220 determines whether or not the safety factor calculated in step S302 is “1.0” or more (step S304). In this case, when less than “1.0”, the safety factor can be said to conform to the actual situation of the slope to be evaluated. Thus, when the safety factor is “1.0” or more (step S304: YES), the data processing unit 220 corrects the influence of vegetation (step S306). For example, in this instance, the data processing unit 220 corrects the values of the adhesion cr and the upper load q0 in such a way that a value of a safety factor to be calculated is lower. When the safety factor is less than “1.0” in step S304 (step S304: NO), the data processing unit 220 skips the processing in step S306.

On the other hand, when a slope failure has not occurred (step S303: NO), the data processing unit 220 determines whether or not the safety factor calculated in step S302 is less than “1.0” (step S305). In this case, when “1.0” or more, the safety factor can be said to conform to the actual situation of the slope to be evaluated. Thus, when the safety factor is less than “1.0” (step S305: YES), the data processing unit 220 corrects the influence of vegetation (step S306). For example, in this instance, the data processing unit 220 corrects the values of the adhesion cᵣ and the upper load q₀ in such a way that a value of a safety factor to be calculated is higher. When the safety factor is “1.0” or more in step S305 (step S305: NO), the data processing unit 220 skips the processing in step S306.

### Modification Example 3

The information processing device 100 may further include other configurations in addition to the configuration described in the first example embodiment. Similarly, the evaluation device 200 may further include other configurations in addition to the configuration described in the second example embodiment. Moreover, the information processing device 100 or the evaluation device 200 may be realized by cooperation of a plurality of devices.

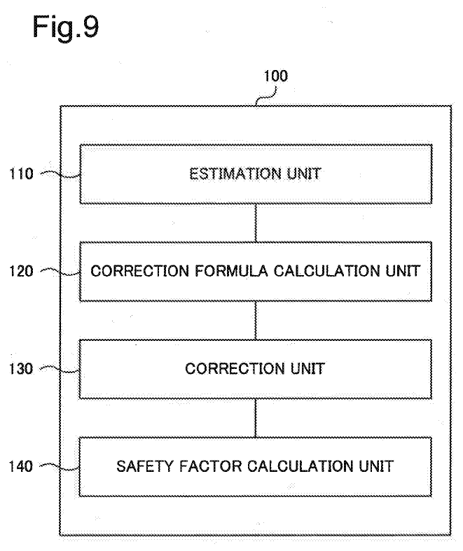

FIG. 9 is a block diagram illustrating one example of another configuration of the information processing device 100. In this example, the information processing device 100 includes a safety factor calculation unit 140 in addition to the estimation unit 110, the correction formula calculation unit 120, and the correction unit 130 similar to those in the first example embodiment. The safety factor calculation unit 140 has, for example, a configuration similar to that of the safety factor calculation unit 230 according to the second example embodiment. Note that, the information processing device 100 may further include a configuration equivalent to the output unit 240 according to the second example embodiment.

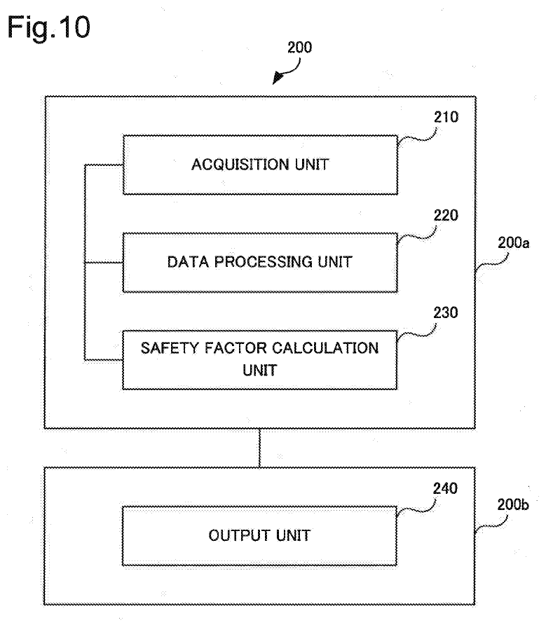

FIG. 10 is a block diagram illustrating one example of another configuration of the evaluation device 200. In this example, the evaluation device 200 is configured by a first module 200a including the acquisition unit 210, the data processing unit 220, and the safety factor calculation unit 230, and a second module 200b including the output unit 240. The first module 200a and the second module 200b may have different main bodies of operation. For example, the first module 200a and the second module 200b are different instruments connected in a wired or wireless manner.

Note that, the acquisition unit 210 needs only to include at least one or more of the topography data acquisition unit 211, the vegetation data acquisition unit 212, and the geology data acquisition unit 213 illustrated in FIG. 5. In other words, the first data need only to include at least one or more of topography data, vegetation data, and geology data.

### Modification Example 4

Specific hardware configurations of the information processing device 100 and the evaluation device 200 include various possible variations, and are not limited to particular configurations. For example, some of the components of the information processing device 100 and the evaluation device 200 may be realized by use of software.

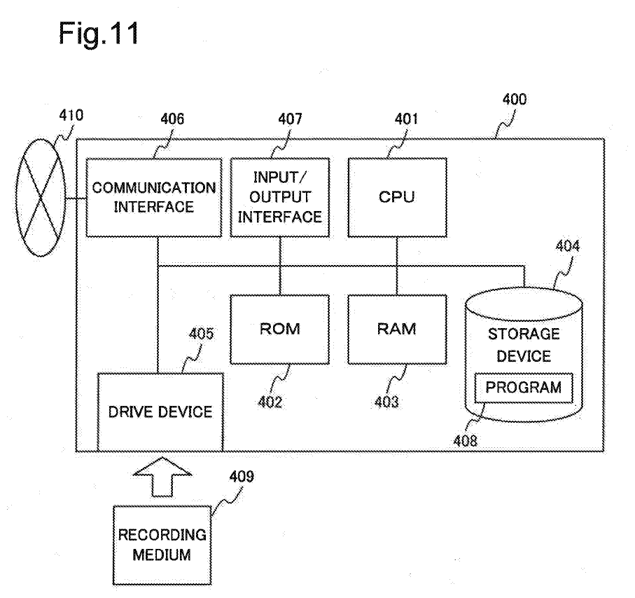

FIG. 11 is a block diagram illustrating one example of a hardware configuration of a computer device 400 realizing the information processing device 100 or the evaluation device 200. The computer device 400 includes a central processing unit (CPU) 401, a read only memory (ROM) 402, a random access memory (RAM) 403, a storage device 404, a drive device 405, a communication interface 406, and an input/output interface 407. The information processing device 100 and the evaluation device 200 can be realized by the configuration (or a part thereof) illustrated in FIG. 11.

The CPU 401 executes a program 408 by use of the RAM 403. The program 408 may be stored in the ROM 402. Alternatively, the program 408 may be recorded in a recording medium 409 such as a flash memory, and read by the drive device 405, or transmitted from an external device via a network 410. The communication interface 406 exchanges data with the external device via the network 410. The input/output interface 407 exchanges data with peripheral equipment (an input device, a display device, and the like). The communication interface 406 and the input/output interface 407 are able to function as a means for acquiring or outputting data.

Note that, the information processing device 100 and the evaluation device 200 may be each configured by a single circuitry (processor or the like), or configured by a combination of plurality of circuitries. The circuitry referred to herein may be either a dedicated circuitry or a general-purpose circuitry. Alternatively, the information processing device 100 or the evaluation device 200 may be configured by a single circuitry.

[Supplementary Notes]

Some or all of the example embodiments of the present invention may also be described as in Supplementary notes below, but are not limited to the followings.

(Supplementary Note 1)

An information processing device comprising:

estimation means for estimating a parameter indicating a moisture state of soil at a predetermined site, based on first data indicating topography, vegetation, or geology of the site, and second data indicating a precipitation amount at the site;

correction formula calculation means for calculating a correction formula, in regard to a first site where a sensor that measures the parameter is installed, by using a parameter measured by the sensor and a first parameter being estimated for the first site; and

correction means for correcting, by using the calculated correction formula, a second parameter estimated for a second site where a sensor that measures the parameter is not installed.

(Supplementary Note 2)

The information processing device according to Supplementary note 1, wherein

the estimation means estimates the first parameter and the second parameter by simulating surface movement of water and underground movement of water at a site corresponding to each parameter, respectively.

(Supplementary Note 3)

The information processing device according to Supplementary note 1 or 2, wherein

a plurality of the first sites exist,

the estimation means estimates the first parameter for each of a plurality of the first sites, and

the correction formula calculation means calculates the correction formula for each of a plurality of the first sites.

(Supplementary Note 4)

The information processing device according to Supplementary note 3, wherein

the correction means corrects the second parameter for the second site, by using a correction formula calculated for a site close in distance to the second site among a plurality of the first sites.

(Supplementary Note 5)

The information processing device according to Supplementary note 3 or 4, wherein

the correction means corrects the second parameter for the second site, by using a correction formula calculated for a site similar in at least one of topography, vegetation, and geology to the second site among a plurality of the first sites.

(Supplementary Note 6)

The information processing device according to any one of Supplementary notes 3 to 5, wherein

the correction formula calculation means calculates a weighted correction formula using a plurality of correction formulas calculated for a plurality of the first sites, and the correction means corrects the second parameter by using the weighted correction formula.

(Supplementary Note 7)

The information processing device according to Supplementary note 6, wherein

the correction formula calculation means varies a weight in the weighted arithmetical operation, depending on distances between a plurality of the first sites and the second site.

(Supplementary Note 8)

The information processing device according to Supplementary note 6 or 7, wherein the correction formula calculation means varies a weight for the correction formula, depending on a difference in at least one of topography, vegetation, and geology between a plurality of the first sites and the second site.

(Supplementary Note 9)

The information processing device according to any one of Supplementary notes 1 to 8, further comprising safety factor calculation means for calculating a safety factor at the second site, based on the corrected second parameter.

(Supplementary Note 10)

A parameter correction method comprising:

estimating a parameter indicating a moisture state of soil at a predetermined site, based on first data indicating topography, vegetation, or geology of the site, and second data indicating a precipitation amount at the site;

calculating a correction formula, in regard to a first site where a sensor that measures the parameter is installed, by using a parameter measured by the sensor and a first parameter estimated for the first site; and

correcting, by using the calculated correction formula, a second parameter estimated for a second site where a sensor that measures the parameter is not installed.

(Supplementary Note 11)

A computer-readable program recording medium recording a program causing a computer to execute:

processing of estimating a parameter indicating a moisture state of soil at a predetermined site, based on first data indicating topography, vegetation, or geology of the site, and second data indicating a precipitation amount at the site;

processing of calculating a correction formula, in regard to a first site where a sensor that measures the parameter is installed, by using a parameter measured by the sensor and a first parameter estimated for the first site; and

processing of correcting, by using the calculated correction formula, a second parameter estimated for a second site where a sensor that measures the parameter is not installed.

The present invention has been described so far with the above example embodiments as an exemplar. However, the present invention is not limited to the example embodiments described above. In other words, various aspects that can be appreciated by a person skilled in the art can be applied to the present invention within the scope of the present invention.

This application is based upon and claims the benefit of priority from Japanese patent application No. 2016-031721, filed on Feb. 23, 2016, the disclosure of which is incorporated herein in its entirety by reference.

### REFERENCE SIGNS LIST

100 Information processing device
110 Estimation unit
120 Correction formula calculation unit
130 Correction unit
140 Safety factor calculation unit
20 Evaluation system
200 Evaluation device
210 Acquisition unit
220 Data processing unit
230 Safety factor calculation unit
240 Output unit
300 Soil sensor

1. An information processing device comprising:
estimation means for estimating a parameter indicating a moisture state of soil at a predetermined site, based on first data indicating topography, vegetation, or geology of the site, and second data indicating a precipitation amount at the site;
correction formula calculation means for calculating a correction formula, in regard to a first site where a sensor that measures the parameter is installed, by using a parameter measured by the sensor and a first parameter being estimated for the first site; and
correction means for correcting, by using the calculated correction formula, a second parameter estimated for a second site where a sensor that measures the parameter is not installed.

2. The information processing device according to claim 1, wherein
the estimation means estimates the first parameter and the second parameter by simulating surface movement of water and underground movement of water at a site corresponding to each parameter, respectively.

3. The information processing device according to claim 1, wherein
a plurality of the first sites exist,
the estimation means estimates the first parameter for each of a plurality of the first sites, and
the correction formula calculation means calculates the correction formula for each of a plurality of the first sites.

4. The information processing device according to claim 3, wherein
the correction means corrects the second parameter for the second site, by using a correction formula calculated for a site close in distance to the second site among a plurality of the first sites.

5. The information processing device according to claim 3, wherein
the correction means corrects the second parameter for the second site, by using a correction formula calculated for a site similar in at least one of topography, vegetation, and geology to the second site among a plurality of the first sites.

6. The information processing device according to any one of claims 3, wherein
the correction formula calculation means calculates a weighted correction formula using a plurality of correction formulas calculated for a plurality of the first sites, and
the correction means corrects the second parameter by using the weighted correction formula.

7. The information processing device according to claim 6, wherein
the correction formula calculation means varies a weight in the weighted arithmetical operation, depending on distances between a plurality of the first sites and the second site.

8. The information processing device according to claim 6, wherein
the correction formula calculation means varies a weight for the correction formula, depending on a difference in at least one of topography, vegetation, and geology between a plurality of the first sites and the second site.

9. The information processing device according to any one of claims 1, further comprising
safety factor calculation means for calculating a safety factor at the second site, based on the corrected second parameter.

10. A parameter correction method comprising:
estimating a parameter indicating a moisture state of soil at a predetermined site, based on first data indicating topography, vegetation, or geology of the site, and second data indicating a precipitation amount at the site;
calculating a correction formula, in regard to a first site where a sensor that measures the parameter is installed, by using a parameter measured by the sensor and a first parameter estimated for the first site; and
correcting, by using the calculated correction formula, a second parameter estimated for a second site where a sensor that measures the parameter is not installed.

11. A computer-readable program recording medium recording a program causing a computer to execute:
processing of estimating a parameter indicating a moisture state of soil at a predetermined site, based on first data indicating topography, vegetation, or geology of the site, and second data indicating a precipitation amount at the site;
processing of calculating a correction formula, in regard to a first site where a sensor that measures the parameter is installed, by using a parameter measured by the sensor and a first parameter estimated for the first site; and
processing of correcting, by using the calculated correction formula, a second parameter estimated for a second site where a sensor that measures the parameter is not installed.
